(19) United States
(12) Patent Application Publication
(10) Pub. No.: US 2020/0140375 A1
(43) Pub. Date: May 7, 2020

(54) COMPOSITIONS AND METHODS FOR TREATING CONDITIONS ASSOCIATED WITH ALTERED TCA CYCLE METABOLISM

(71) Applicant: IMBRIA PHARMACEUTICALS, INC., Rancho Santa Fe, CA (US)

(72) Inventor: Andrew D. Levin, Newton, MA (US)

(73) Assignees: IMBRIA PHARMACEUTICALS, INC., Rancho Santa Fe, CA (US); IMBRIA PHARMACEUTICALS, INC., Rancho Santa Fe, CA (US)

(21) Appl. No.: 16/324,406

(22) PCT Filed: Jul. 24, 2018
(86) PCT No.: PCT/US2018/043487

§ 371 Date: Feb. 8, 2019

Related U.S. Application Data
(60) Provisional application No. 62/677,940, filed on May 30, 2018.
(60) Provisional application No. 62/667,893, filed on May 7, 2018.
(60) Provisional application No. 62/662,014, filed on Apr. 24, 2018.
(60) Provisional application No. 62/650,395, filed on Mar. 30, 2018.
(60) Provisional application No. 62/710,357, filed on Feb. 16, 2018.
(60) Provisional application No. 62/547,547, filed on Aug. 18, 2017.
(60) Provisional application No. 62/536,318, filed on Jul. 24, 2017.

(51) Int. Cl. C07C 229/22; C07C 229/36; C07C 321/04

(52) U.S. Cl. CPC C07C 229/22; C07C 69/40; C07C 321/04; C07C 229/36

(57) ABSTRACT

Compositions and methods for treating conditions associated with altered TCA cycle metabolism are provided.

### CROSS-REFERENCE TO RELATED APPLICATIONS

This application claims the benefit of, and priority to, U.S. Provisional Patent Application No. 62/677,940, filed May 30, 2018, U.S. Provisional Patent Application No. 62/667,893, filed May 7, 2018. U.S. Provisional Patent Application No. 62/662,014, filed Apr. 24, 2018. U.S. Provisional Patent Application No. 62/650,395, filed Mar. 30, 2018, U.S. Provisional Patent Application No. 62/710,357, filed Feb. 16, 2018, U.S. Provisional Patent Application No. 62/547,547, filed Aug. 18, 2017, and U.S. Provisional Patent Application No. 62/536,318, filed Jul. 24, 2017, the contents of each of which are incorporated by reference.

### FIELD OF THE INVENTION

This application is related to compositions and methods for treating conditions associated with altered TCA cycle metabolism.

### BACKGROUND

Abnormal metabolism of the tricarboxylic acid (TCA) cycle (also known as the citric acid cycle or Krebs cycle) is associated with a variety of diseases, including inherited metabolic disorders, neurodegenerative diseases, and cancers. Inherited disorders of the TCA cycle cause intellectual disability, various neurological problems, and death in young children, while the neurodegenerative diseases and cancers that are coupled to dysfunction of the TCA cycle lead to cognitive and physical disabilities and death in adults.

Although the TCA cycle and its relationship to other intermediary metabolic pathways have been understood for decades, effective therapies for treating conditions associated with abnormal TCA cycle metabolism are lacking. Efforts to develop compositions that restore TCA cycle metabolism by delivering TCA cycle metabolites have been unsatisfactory. Compounds that provide unadulterated TCA cycle intermediates are challenging to administer orally due to the large amount of material that is needed to be taken by mouth and strong tastes or odors. Existing compositions are inadequate to remedy dysfunction of the TCA cycle, and people continue to suffer and die from a variety of conditions related to abnormal TCA cycle metabolism.

### SUMMARY

The invention overcomes the challenges of administering large quantities of TCA intermediates by incorporating such intermediates into higher solubility molecules that can be metabolized to release the intermediates in the body. The invention recognizes that conjugating amino acids to TCA cycle intermediates dramatically increases the solubility of those compounds. With improved solubility, the compounds can be administered orally at the high doses needed to treat conditions associated with altered TCA cycle metabolism.

In that manner, the invention provides compositions that contain one or more TCA cycle intermediates conjugated to one or more amino acids. The conjugates are highly soluble in water and can be cleaved in the body to release the TCA cycle intermediate for efficient delivery to target tissues. Preferably, the TCA cycle intermediate is succinate, and the amino acid is serine or tyrosine. The conjugates may include multiple TCA intermediate-amino acid moieties linked by a polyol, such as a C₂-C₂₀ polyol, as, for example, in glycerol trisuccinate triserine. The invention also provides methods of treating a condition associated with altered TCA cycle metabolism by providing compositions of the invention.

Because the compositions provide TCA cycle intermediates in water-soluble compounds, they are useful as therapeutic agents for treating conditions associated with abnormal TCA cycle metabolism. Due to the high solubility of the compounds, they are readily absorbed, circulate throughout the body and can be cleaved to make the TCA cycle intermediate available to target tissues. In addition, the compounds are suitable for oral administration because the covalent linkage eliminates the taste or odor produced by free TCA cycle intermediates. Thus, the compositions of the invention also result in better patient compliance with a therapeutic regimen compared to formulations that use free TCA cycle intermediates.

In an aspect, the invention provides compounds that include one or more TCA cycle intermediates or prodrugs thereof and one or more amino acids. The compounds may include two or more TCA cycle intermediates or prodrugs thereof chemically linked to each other. The compounds may include two or more amino acids.

Numerous TCA cycle intermediates or prodrugs thereof are known in the art (such as those described in PCT/US2017/019000, the content of which is incorporated by reference herein in its entirety. Any such compounds may be conjugated with one or more amino acids to improve the solubility, and therefore oral availability of those compounds. In certain embodiments, the TCA cycle intermediate or prodrug thereof may be citrate, cis-aconitate. D-isocitrate, α-ketoglutarate, succinate, fumarate, malate, oxaloacetate, acetone, acetoacetate, β-hydroxybutyrate, β-ketopentanoate, or β-hydroxypentanoate. Preferably, the TCA cycle intermediate is succinate.

The TCA cycle intermediate or prodrug may include succinate diserine, glycerol trisuccinate triserine, or glycerol trisuccinate trityrosine. The TCA cycle intermediate or prodrug may include a structure represented by one of formulas (I), (II) and (III):

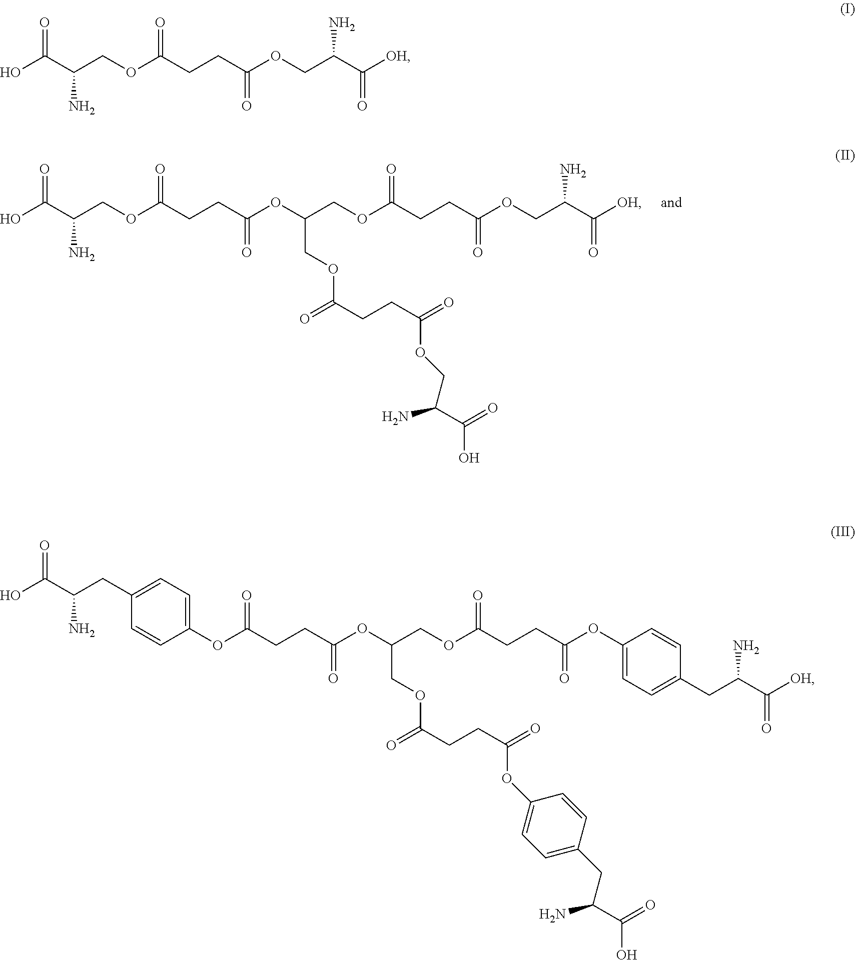 [SMILES: N[C@@H](COC(=O)CCC(=O)OC[C@H](N)C(=O)O)C(=O)O ; N[C@@H](COC(=O)CCC(=O)OCC(COC(=O)CCC(=O)OC[C@H](N)C(=O)O)OC(=O)CCC(=O)OC[C@H](N)C(=O)O)C(=O)O ; N[C@@H](Cc1ccc(OC(=O)CCC(=O)OCC(COC(=O)CCC(=O)Oc2ccc(C[C@H](N)C(=O)O)cc2)OC(=O)CCC(=O)Oc2ccc(C[C@H](N)C(=O)O)cc2)cc1)C(=O)O]

The TCA cycle intermediate or prodrug may include a structure represented by formula (IV):

A-β-hydroxybutyrate-B-β-hydroxybutyrate-A (IV),

in which A is an amino acid and B is a TCA cycle intermediate. In preferred embodiments, A is serine, and B is succinate.

The TCA cycle intermediate or prodrug may include a structure represented by formula (V):

C-D-E (V),

in which C is a first TCA cycle intermediate, D is a second TCA cycle intermediate, and E is an amino acid. In preferred embodiments, C is malate, D is succinate, and E is serine.

The amino acid may be any naturally-occurring or non-naturally-occurring amino acid. The amino acid may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. The amino acid may be serine and tyrosine. Preferably, the amino acid is serine. In compounds that include two or more amino acids, the two or more amino acids may be the same, i.e., multiple copies of the same molecular species, or they may be different.

In compounds that include two or more TCA intermediates or prodrugs thereof, the two or more TCA intermediates or prodrugs thereof may be the same, i.e., multiple copies of the same molecular species, or different. The two or more TCA intermediates or prodrugs thereof may be attached directly to each other, or they may be attached via a linker. The two or more TCA intermediates or prodrugs thereof may be linked via a polyol, such as a C₂-C₂₀ polyol. Preferably, the two or more TCA intermediates or prodrugs thereof are linked via glycerol, erythritol, or xylitol. The compounds may be represented by formula (VI):

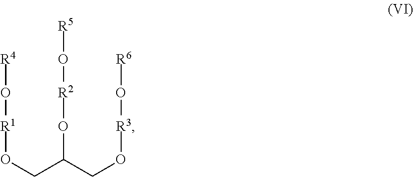

in which R¹, R², and R³ are TCA cycle intermediates or prodrugs thereof, and R⁴, R⁵, and R⁶ are amino acids. R¹, R², and R³ may be the same or different, and R⁴, R⁵, and R⁶ may be the same or different. R¹, R², and R³ may be succinate. R⁴, R⁵, and R⁶ may be serine, threonine, or tyrosine. If R⁴, R⁵, and R⁶ are serine, threonine, or tyrosine, they may be linked via the oxygen atom on their side chains, and the carboxyl group and amino group may be free and thus able to form COO⁻ and NH₃⁺ ions in aqueous solutions.

Any of the compounds described above may include one or more atoms that are enriched for an isotope. For example, the compounds may have one or more hydrogen atoms replaced with deuterium or tritium. The isotopically enriched atom or atoms may be located at any position within the compound.

In certain embodiments, the compounds may have an octanol:water partition coefficient of less than 0.1, less than 0.01, less than 0.001, less than 0.0001, less than 0.0001, less than 0.00001, or less than 0.000001.

In certain embodiments, the compounds include a polyol, a TCA cycle intermediate or prodrug thereof covalently linked to the polyol, and an amino acid covalently linked to the TCA cycle intermediate or prodrug thereof. Each of the polyol, the TCA cycle intermediate or prodrug thereof, and the amino acid may be as described above in reference to such components. Preferably, the polyol is glycerol, the TCA intermediates or prodrugs thereof is succinate, and the amino acid is serine. The polyol may be linked via a terminal hydroxy group or an internal hydroxy group. For example, glycerol may linked to the TCA cycle intermediate or prodrug thereof via a hydroxy group on its first, second, or third carbon. The compound may be represented by one of formulas (XIII) and (XIV):

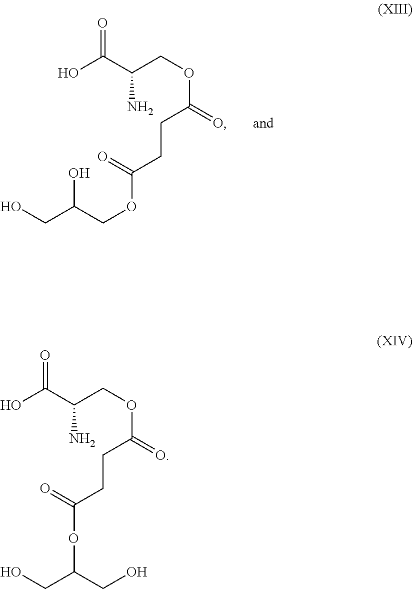

In certain embodiments, the TCA cycle intermediate is α-ketoglutarate. Optionally, the amino acid is serine. Optionally, the polyol is glycerol. In certain embodiments, the compound is represented formula (XV):

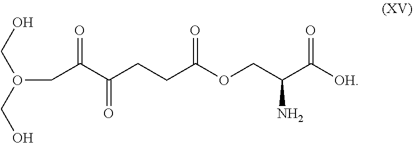

In other embodiments, the TCA cycle intermediate is 1-hydroxybutyrate. Optionally, the amino acid is serine. Optionally, the polyol is glycerol. In certain embodiments, the compound is represented formula (XVI):

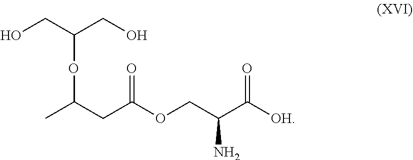 [SMILES: CC(CC(=O)OC[C@H](N)C(=O)O)OC(CO)CO]

In another aspect, the invention provides methods of treating a condition associated with altered TCA cycle metabolism in a subject. The methods include providing the subject with a composition of the invention, as described above.

The condition associated with altered TCA cycle metabolism may be an inherited disorder, such as 2-oxoglutaric aciduria, fumarase deficiency, or succinyl-CoA synthetase deficiency. The condition associated with altered TCA cycle metabolism may be a neurodegenerative disorder, such as Amyotrophic Lateral Sclerosis. Alzheimer's disease, Parkinson's disease, or Huntington's disease. The condition associated with altered TCA cycle metabolism may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma.

The condition associated with altered TCA cycle metabolism may be an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy (e.g., Duchenne's muscular dystrophy and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.

The solubility of TCA cycle intermediates can be increased by covalently linking capping moieties to such molecules. Thus, in another aspect, the invention provides compounds that include a TCA cycle intermediate or prodrug thereof and covalently linked to two or more capping moieties. For example, the compounds may include a TCA cycle intermediate linked to two, three, four, five, or six capping moieties.

The TCA cycle intermediate or prodrug thereof may be citrate, cis-aconitate, D-isocitrate, α-ketoglutarate, succinate, fumarate, malate, oxaloacetate, acetone, acetoacetate, β-hydroxybutyrate, β-ketopentanoate, or β-hydroxypentanoate. Preferably, the TCA cycle intermediate is succinate. The TCA cycle intermediate may have L or R chirality. Compositions including such compounds may include only L-forms, only R-forms, or racemic mixtures of L- and R-forms of the TCA cycle intermediate.

The two or more capping moieties may be the same, or they may be different. The capping moieties may be polyols, such as C₂-C₂₀ polyols, amino acids, or other TCA cycle intermediates or prodrugs thereof. The compound may have two capping moieties, both of which are glycerol. The compound may have two capping moieties, with one being malate and the other being serine.

The capping moieties may be linked by any atoms on the TCA cycle intermediate or prodrug thereof. Preferably, capping moieties are substituted onto hydroxyl groups and attached via alkoxy linkages. Preferably, a capping moiety is substituted onto the hydroxyl group of each of the terminal carbon atoms in the carbon skeleton of the TCA cycle intermediate or prodrug thereof. The TCA cycle intermediate or prodrug thereof may be represented by one of formulas (VII), (VIII), (IX), (X), (XI), and (XII):

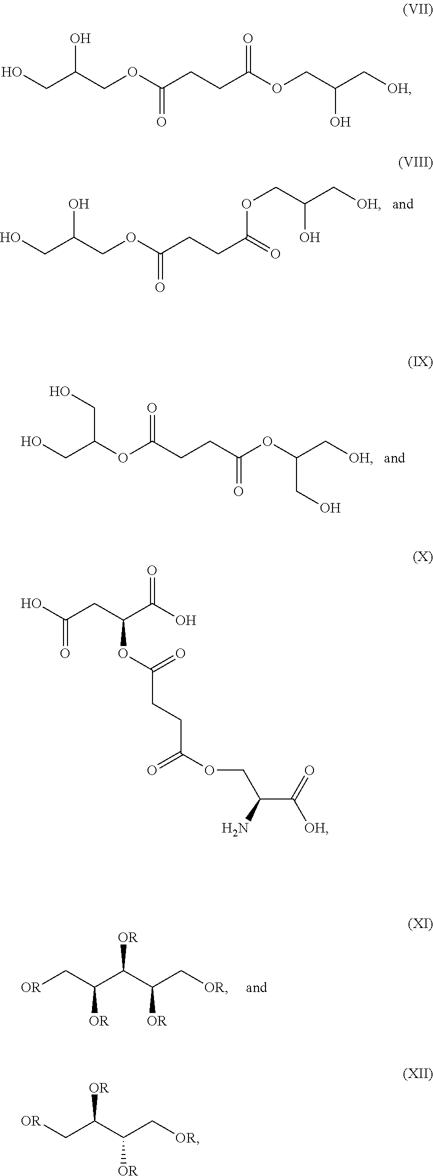

in which R is

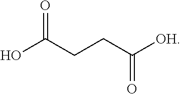

The capping moiety may include one or more atoms that are enriched for an isotope. For example, the capping moiety may have one or more hydrogen atoms replaced with deuterium or tritium. The isotopically enriched atom or atoms may be located at any position within the capping moiety.

In other embodiments, the invention provides compounds including citrate or citric acid, prodrugs, analogs, derivatives, or salts thereof, and one or more amino acids. In certain embodiments, the compound includes a plurality of amino acids, e.g., at least two or three amino acids. In preferred embodiments, the compound includes three amino acids. Numerous different types of amino acids can be conjugated to the citrate. The amino acids may be any naturally-occurring or non-naturally-occurring amino acids or combinations thereof (e.g., all naturally occurring, all non-naturally occurring, or a combination of naturally and non-naturally occurring amino acids). The amino acids may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. The amino acid may be serine and tyrosine. In certain embodiments, the amino acid is serine and the compound includes three serines. An exemplary compound is represented formula (XVII):

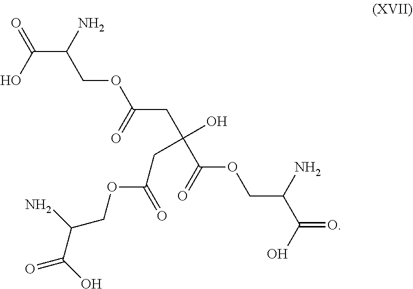 [SMILES: NC(COC(=O)CC(O)(CC(=O)OCC(N)C(=O)O)C(=O)OCC(N)C(=O)O)C(=O)O]

In another aspect, the invention provides compositions comprising at least one TCA cycle intermediate or prodrug thereof covalently bound to one or more polyol molecules in a therapeutically effective amount to treat a condition associated with altered TCA cycle metabolism in a subject. The invention encompasses various ratios of the at least one TCA cycle intermediate or prodrug and the one or more polyol molecules. Exemplary ratios include 1:1, 2:1, 3:1, 1:2, or 1:3. A preferred ratio is 1:1. In certain embodiments, the composition is formulated for oral administration. In certain embodiments, the composition is formulated as a single unit dose.

In certain embodiments, the TCA cycle intermediate or prodrug thereof is selected from the group consisting of citrate, cis-aconitate, D-isocitrate, α-ketoglutarate, succinate, fumarate, malate, oxaloacetate, pyruvate, acetone, acetoacetate, β-hydroxybutyrate, β-ketopentanoate, and β-hydroxypentanoate. In particular embodiments, the TCA cycle intermediate or prodrug thereof is citrate. In certain embodiments, the polyol is glycerol. In certain of such embodiments, the composition comprises a plurality of citrate molecules covalently bound to one or more glycerol molecules. In a preferred embodiment, the composition comprises a plurality of citrate molecules, at least one of which is covalently bound to a plurality of glycerol molecules. The invention encompasses various ratios of citrate and the one or more polyol molecules. Exemplary ratios include 1:1, 2:1, 3:1, 1:2, or 1:3. A preferred ratio is 1:1. A preferred compound is a compound of Formula XVIII:

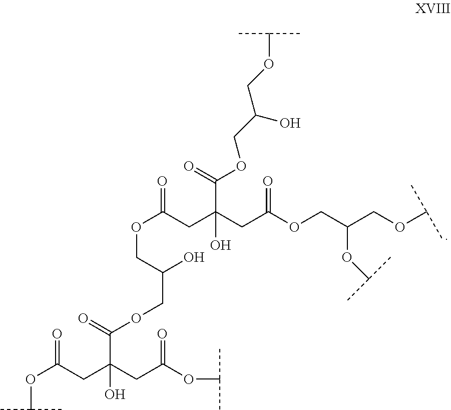

Another aspect of the invention provides methods for treating a condition associated with altered TCA cycle metabolism in a subject comprising providing any of the above described compositions in a therapeutically effective amount to treat the condition associated with altered TCA cycle metabolism in the subject. The condition associated with altered TCA cycle metabolism may be an inherited disorder, a neurodegenerative disorder, a cancer, an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy, mitochondrial myopathy, mitochondrial encephalomyopathy lactic acidosis and stroke-like syndrome (MELAS), myoclonic epilepsy and ragged-red fibers (MERRF), a mitochondrial associated disease, and a disorder related to POLG mutation.

In another aspect, the invention provides compositions comprising a TCA cycle intermediate anhydride or polymer, or pharmaceutically acceptable salt or prodrug thereof in a therapeutically effective amount to treat a condition associated with altered TCA cycle metabolism in a subject. The TCA cycle intermediate or polymer or pharmaceutically acceptable salt or prodrug thereof may be selected from the group consisting of citrate, cis-aconitate, D-isocitrate, α-ketoglutarate, succinate, fumarate, malate, oxaloacetate, pyruvate, acetone, acetoacetate, β-hydroxybutyrate. β-ketopentanoate, and β-hydroxypentanoate.

In certain embodiments, the prodrug comprises one or more polyols. In other embodiments, the prodrug comprises one or more amino acids. In certain embodiments, the prodrug comprises one or more polyols and one or more amino acids. In certain embodiments, the polymer form is a repeating unit of the TCA cycle intermediate anhydride.

In an exemplary embodiment, the TCA cycle intermediate anhydride or polymer or pharmaceutically acceptable salt or prodrug thereof is citric acid anhydride or a pharmaceutically acceptable salt or prodrug thereof. In certain embodiments, the citric acid anhydride is a prodrug of citric acid anhydride. Such exemplary prodrugs may comprise one or more polyols. In other embodiments, the citric acid anhydride prodrug comprises one or more amino acids. In certain embodiments, the citric acid anhydride prodrug comprises one or more polyols and one or more amino acids. In certain embodiments, the citric acid anhydride is selected from the group consisting of a symmetrical citric acid anhydride, an asymmetrical citric acid anhydride, an intermolecular citric acid anhydride, and a combination thereof. In certain embodiments, the composition is a citric acid anhydride polymer, e.g., repeating units of a citric acid anhydride monomer unit linked together.

In certain embodiments, the composition is formulated for oral or gastric administration. In certain embodiments, the composition is formulated as a single unit dose.

Another aspect of the invention provides methods for treating a condition associated with altered TCA cycle metabolism in a subject comprising providing any of the above described compositions in a therapeutically effective amount to treat the condition associated with altered TCA cycle metabolism in the subject. The condition associated with altered TCA cycle metabolism may be an inherited disorder, a neurodegenerative disorder, a cancer, an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy, mitochondrial myopathy, mitochondrial encephalomyopathy lactic acidosis and stroke-like syndrome (MELAS), myoclonic epilepsy and ragged-red fibers (MERRF), a mitochondrial associated disease, and a disorder related to POLG mutation.

### DETAILED DESCRIPTION

The invention provides compositions that allow efficient delivery of TCA cycle intermediates for treatment of conditions associated with abnormal TCA cycle metabolism. Such intermediates are conjugated to amino acids in compounds that are highly soluble in water to facilitate system absorption and circulation. In addition, the compounds can be cleaved in the body to release the intermediates in a form that enter the cycle either directly or by conversion through intermediary metabolic pathways.

The TCA cycle is illustrated below:

Abnormal TCA cycle metabolism is associated with a variety of conditions. In hereditary metabolic disorders of the TCA cycle, such as 2-oxoglutaric aciduria, fumarase deficiency, and succinyl-CoA synthetase deficiency, genetic mutations affect enzymes of the TCA cycle or enzymes that catalyze related reactions. Consequently, individual reactions of the TCA cycle are impaired, leading to the depletion of intermediates required for the cycle to proceed. Such diseases typically present early with severe symptoms, such as mental retardation, microcephaly, deafness, and hypotonia and are often fatal in early childhood.

Abnormal TCA cycle metabolism is also observed in other diseases that do not have direct genetic links to this metabolic pathway. For example, altered TCA metabolism is observed in neurodegenerative disorders, such as Amyotrophic Lateral Sclerosis, Alzheimer's disease, Parkinson's disease, or Huntington's disease, and in a wide variety of cancers. Although the symptoms this diverse set of diseases vary, in many cases decreased activity of specific TCA enzymes or decreased mitochondrial ATP production has been observed, and it is believed that boosting levels of TCA cycle intermediates would mitigate the symptoms and improve prognoses.

Efforts have been made to identify compounds that can be used in oral formulations for delivery of TCA cycle intermediates to remedy metabolic deficiencies. For example, compounds that contain a glycerol backbone linked to both succinate and fatty acids are disclosed in PCT/US2017/019000, which is incorporated herein by reference. However, such compounds are lipophilic and poorly soluble in water, which limits their bioavailability.

The compounds provided herein overcome the limited bioavailability of previously described compositions for delivery of TCA cycle intermediates. Because the compounds of the invention are highly water soluble, they are absorbed and circulate readily in the body. In addition, the compounds can be cleaved to efficiently deliver TCA cycle intermediates to target tissues. Due to their superior bioavailability, the compounds of the invention can be provided in doses suitable for oral administration to treat abnormal TCA metabolism associated with a wide range of conditions.

The compounds of the invention include (1) one or more TCA cycle intermediates, metabolites that feed into the TCA cycle, such as pyruvate or ketone bodies, or prodrugs of TCA cycle intermediates or metabolites that feed into the TCA cycle and (2) one or more amino acids. Any of the TCA cycle intermediates described in the TCA cycle above may be used in compositions of the invention. In certain embodiments, any of the compounds described in PCT/US2017/019000 may be TCA cycle intermediates within the context of the invention.

A prodrug is a medication or compound that, after administration, is metabolized (i.e., converted within the body) into a pharmacologically active drug. The prodrug itself may be pharmacologically inactive. Prodrugs may be used to improve how a medicine is absorbed, distributed, metabolized, and excreted. The prodrug may improve the bioavailability of the active drug when the active drug is poorly absorbed from the gastrointestinal tract. The prodrug may improve how selectively the drug interacts with cells or processes that are not its intended target, thereby reducing unintended and undesirable side effects. The prodrug may be converted into a biologically active form (bioactivated) inside cells (a Type I prodrug) or outside cells (a Type II prodrug). The prodrug may bioactivated in the gastrointestinal tract, in systemic circulation, in metabolic tissue other than the target tissue, or in the target tissue.

Thus, the compounds of the invention can be metabolized in the body to yield an intermediate of the TCA cycle, such as citrate, cis-aconitate. D-isocitrate, α-ketoglutarate, succinate, fumarate, malate, or oxaloacetate, or a molecule that can be metabolized to enter the TCA cycle, such as pyruvate or a ketone body. Examples of ketone bodies include acetone, acetoacetate, β-hydroxybutyrate, β-ketopentanoate, or β-hydroxypentanoate.

Any prodrugs of the TCA cycle intermediates described in the TCA cycle above may be used in compositions of the invention. Any of the prodrugs or prodrugs of the compounds described in PCT/US2017/019000 may be TCA cycle intermediate prodrugs within the context of the invention.

The TCA cycle intermediate or prodrug thereof may include one or more substituents. The one or more substituents may be linked via, via any suitable chemical linkage, such as an alkoxyl linkage, to one or more carboxyl groups on the intermediate or prodrug thereof. The substituent may be a short-chain fatty acid, such as formate, acetate, propionate, butyrate, isobutyrate, valerate, or isovalerate.

The TCA cycle intermediate or prodrug may include succinate diserine, glycerol trisuccinate triserine, or glycerol trisuccinate trityrosine. The TCA cycle intermediate or prodrug may include a structure represented by one of formulas (I). (II) and (III):

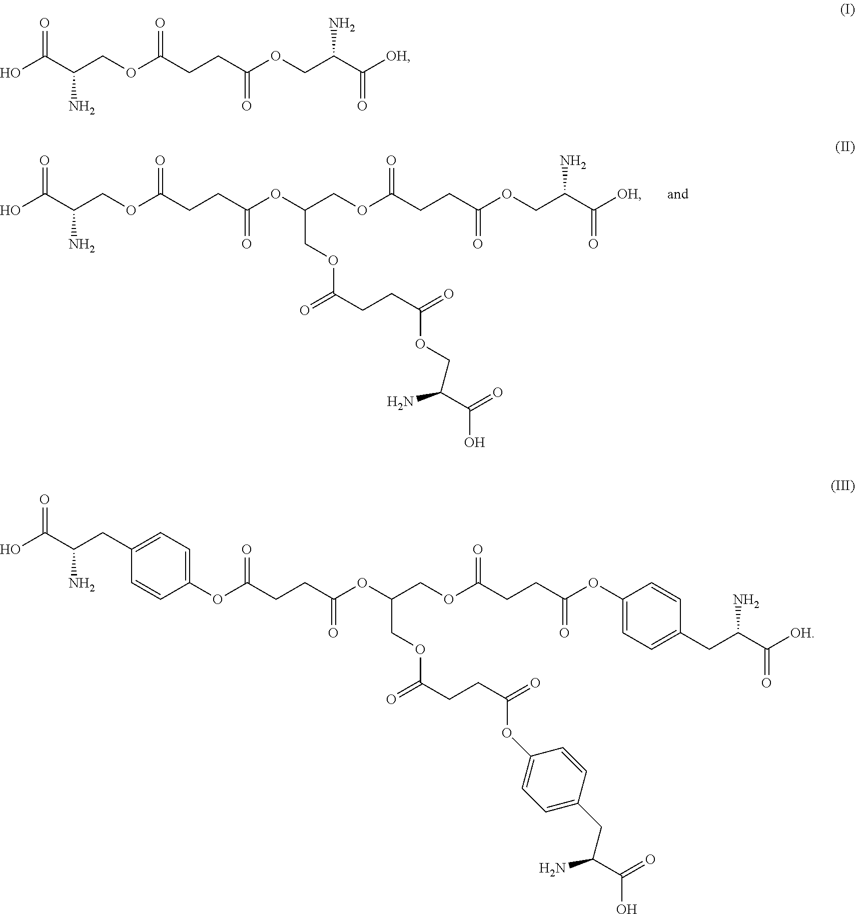

The TCA cycle intermediate or prodrug may include a structure represented by formula (IV):

A-β-hydroxybutyrate-B-β-hydroxybutyrate-A (IV).

in which A is an amino acid and B is a TCA cycle intermediate. In preferred embodiments, A is serine, and B is succinate.

The TCA cycle intermediate or prodrug may include a structure represented by formula (V):

C-D-E (V),

in which C is a first TCA cycle intermediate, D is a second TCA cycle intermediate, and E is an amino acid. In preferred embodiments. C is malate, D is succinate, and E is serine.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)0₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄C(O)R^{∘}; —OC(O)(CH₂)₀₋₄SR^{∘}—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄C(O)NR^{∘}₂—C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR; —(CH₂)0-4OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{●} (or the ring formed by taking two independent occurrences of R^{●} together with their intervening atoms), are independently halogen. —(CH₂)₀₋₂R^{●}, —(haloR^{●}), —(CH₂)0-2OH, —(CH₂)₀₋₂OR^{●}. —(CH₂)₀₋₂CH(OR^{●})₂; —O(haloR^{●}), —CN, —N3, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R^{●} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen. C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, —(haloR^{●}), —OH, —OR^{●}, —O(haloR^{●}), —CN, —C(O)OH, —C(O)OR, —NH2, —NHR^{●}, —NR^{●}, or —NO2, wherein each R^{●} is unsubstituted or where preceded by “halo” is substituted only with one or more halogens, and is independently C₁₋₄ aliphatic. —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen include —R†, —NR†2, —C(O)R†, —C(O)OR†, —C(O)C(O)R†, —C(O)CH₂C(O)R†, —S(O)2R†, —S(O)2NR†₂, —C(S)NR†₂, —C(NH)NR†₂, or —N(R†)S(O)₂R†; wherein each Rt is independently hydrogen, c₁₋₆ aliphatic which may be substituted as defined below, unsubstituted —OPh, or an unsubstituted 5-6-membered saturated, partially unsaturated, oraryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, —(R^{●}), —OH, —OR^{●}, —O(haloR^{●}), —CN, —C(O)OH, —C(O)OR^{●}, —NH₂, —NHR^{●}, —NR^{●}, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The amino acid may be any naturally-occurring or non-naturally-occurring amino acid. Naturally-occurring amino acids include the following twenty amino acids that are encoded by the genetic code and incorporated into polypeptides by the translational machinery: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. Some naturally-occurring amino acids, such as selenocysteine and pyrrolysine, are found in polypeptides but are incorporated by alternative mechanisms. Other naturally-occurring amino acids, such as ornithine, citrulline. β-alanine, carnitine, γ-aminobutyrate, L-thyroxine, hydroxyproline, selenomethionine, and 2-aminoisobutyrate are not found in polypeptides. Non-naturally-occurring amino acids include amino acids that are not found in proteins or produced by cellular metabolic machinery, such as those described in Young and Schultz, Beyond the Canonical 20 Amino Acids: Expanding the Genetic Lexicon, J. Biol. Chem. 285(15): 11039-11044 (2010); U.S. Pat. Nos. 7,566,555; and 9,488,660, each of which is incorporated herein by reference.

The compounds may include two or more TCA intermediates or prodrugs thereof attached via one or more linkers or backbone moieties. The backbone moiety may be a C₂₋₂₀ hydrocarbon moiety substituted with two or more groups selected from one or more of hydroxyl, amino groups, and carboxyl groups. The backbone moiety may be a polyol, such as a C₂-C₂₀ polyol, e.g., glycerol, erythritol, or xylitol. Alternatively or additionally, the two or more TCA intermediates or prodrugs thereof may be attached to each other directly.

The compounds may be represented by formula (VI):

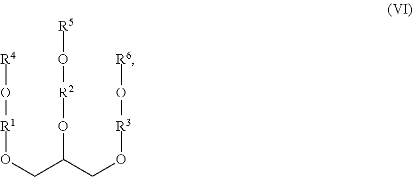

in which R¹, R², and R³ are TCA cycle intermediates or prodrugs thereof, and R⁴, R⁵, and R⁶ are amino acids. R¹, R², and R³ may be the same or different, and R⁴, R⁵, and R⁶ may be the same or different. R¹, R², and R³ may be succinate. R⁴, R⁵, and R⁶ may be serine, threonine, or tyrosine. If R⁴, R⁵, and R⁶ are serine, threonine, or tyrosine, they may be linked via the oxygen atom on their side chains, and the carboxyl group and amino group may be free and thus able to form COO⁻ and NH₃⁺ ions in aqueous solutions.

The compounds, including the capping moieties, may include one or more atoms that are enriched for an isotope. For example, the compounds may have one or more hydrogen atoms replaced with deuterium or tritium. Isotopic substitution or enrichment may occur at carbon, sulfur, or phosphorus atoms as well. The compounds may be isotopically substituted or enriched for a given atom at one or more positions within the compound, or the compounds may be isotopically substituted or enriched at all instances of a given atom within the compound.

The compounds may have an octanol:water partition coefficient of less than 0.1, less than 0.01, less than 0.001, less than 0.0001, less than 0.0001, less than 0.00001, or less than 0.000001.

The solubility of TCA cycle intermediates can be increased by covalently linking capping moieties to such molecules. In particular, it is advantageous to add capping moieties as substituents on the hydroxyl groups of TCA cycle intermediates. Such capped-alcohol molecules have improved solubility and do not have offensive odors.

Thus, in another aspect, the invention provides compounds that include a TCA cycle intermediate or prodrug thereof and covalently linked to two or more capping moieties. For example, the compounds may include a TCA cycle intermediate linked to two, three, four, five, or six capping moieties.

The TCA cycle intermediate or prodrug thereof may be citrate, cis-aconitate, D-isocitrate, α-ketoglutarate, succinate, fumarate, malate, oxaloacetate, acetone, acetoacetate, β-hydroxybutyrate, β-ketopentanoate, or p-hydroxypentanoate. Preferably, the TCA cycle intermediate is succinate. The TCA cycle intermediate may have L or R chirality. Compositions including such compounds may include only L-forms, only R-forms, or racemic mixtures of L- and R-forms of the TCA cycle intermediate.

The two or more capping moieties may be the same, or they may be different. The capping moieties may be polyols, such as C₂-C₂₀ polyols, amino acids, or other TCA cycle intermediates or prodrugs thereof. The compound may have two capping moieties, both of which are glycerol. The compound may have two capping moieties, with one being malate and the other being serine.

The capping moieties may be linked by any atoms on the TCA cycle intermediate or prodrug thereof. Preferably, capping moieties are substituted onto hydroxyl groups and attached via alkoxy linkages. Preferably, a capping moiety is substituted onto the hydroxyl group of each of the terminal carbon atoms in the carbon skeleton of the TCA cycle intermediate or prodrug thereof. The TCA cycle intermediate or prodrug thereof may be represented by one of formulas (VII), (VIII), (IX), (X), (XI), and (XII):

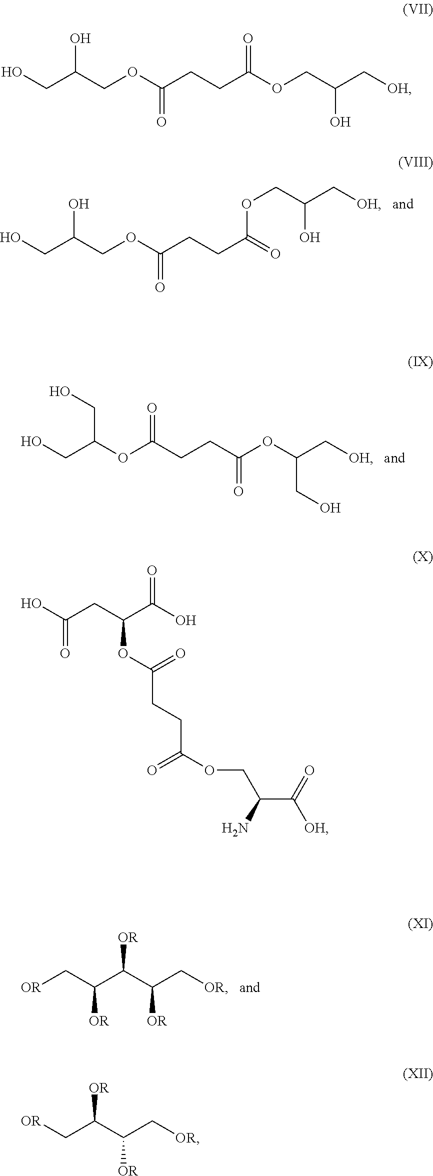

in which R is

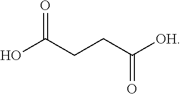

In certain embodiments, the compounds include a polyol, a TCA cycle intermediate or prodrug thereof covalently linked to the polyol, and an amino acid covalently linked to the TCA cycle intermediate or prodrug thereof. Each of the polyol, the CA cycle intermediate or prodrug thereof, and the amino acid may be as described above in reference to such components. Preferably, the polyol is glycerol, the TCA intermediates or prodrugs thereof is succinate, and the amino acid is serine. The polyol may be linked via a terminal hydroxy group or an internal hydroxy group. For example, glycerol may linked to the TCA cycle intermediate or prodrug thereof via a hydroxy group on its first, second, or third carbon. The compound may be represented by one of formulas (XIII) and (XIV):

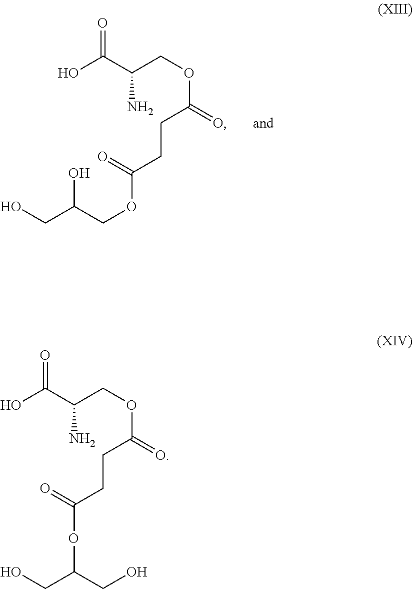

In certain embodiments, the TCA cycle intermediate is α-ketoglutarate. Optionally, the amino acid is serine. Optionally, the polyol is glycerol. In certain embodiments, the compound is represented formula (XV):

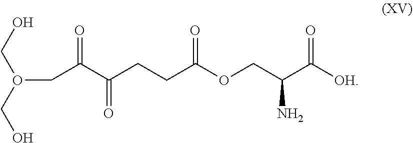

In other embodiments, the TCA cycle intermediate is β-hydroxybutyrate. Optionally, the amino acid is serine. Optionally, the polyol is glycerol. In certain embodiments, the compound is represented formula (XVI):

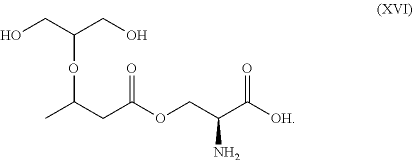 [SMILES: CC(CC(=O)OC[C@H](N)C(=O)O)OC(CO)CO]

In other embodiments, the invention provides compounds including citrate or citric acid, prodrugs, analogs, derivatives, or salts thereof, and one or more amino acids. In certain embodiments, the compound includes a plurality of amino acids, e.g., at least two or three amino acids. In preferred embodiments, the compound includes three amino acids. Numerous different types of amino acids can be conjugated to the citrate. The amino acids may be any naturally-occurring or non-naturally-occurring amino acids or combinations thereof (e.g., all naturally occurring, all non-naturally occurring, or a combination of naturally and non-naturally occurring amino acids). The amino acids may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. The amino acid may be serine and tyrosine. In certain embodiments, the amino acid is serine and the compound includes three serines. An exemplary compound is represented formula (XVII):

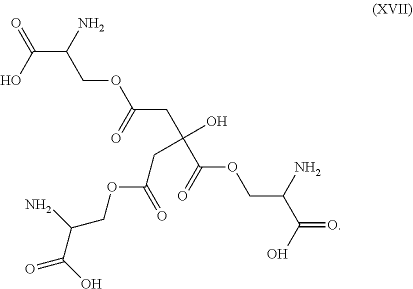 [SMILES: NC(COC(=O)CC(O)(CC(=O)OCC(N)C(=O)O)C(=O)OCC(N)C(=O)O)C(=O)O]

In another aspect, the invention provides composition comprising at least one TCA cycle intermediate or prodrug thereof covalently bound to one or more polyol molecules in a therapeutically effective amount to treat a condition associated with altered TCA cycle metabolism in a subject. In certain embodiments, the composition is formulated for oral administration. In certain embodiments, the composition is formulated as a single unit dose.

In certain embodiments, the TCA cycle intermediate or prodrug thereof is selected from the group consisting of citrate, cis-aconitate, D-isocitrate, α-ketoglutarate, succinate, fumarate, malate, oxaloacetate, pyruvate, acetone, acetoacetate, β-hydroxybutyrate, β-ketopentanoate, and β-hydroxypentanoate. In particular embodiments, the TCA cycle intermediate or prodrug thereof is citrate. In certain embodiments, the polyol is glycerol. In certain of such embodiments, the composition comprises a plurality of citrate molecules covalently bound to one or more glycerol molecules. In a preferred embodiment, the composition comprises a plurality of citrate molecules, at least one of which is covalently bound to a plurality of glycerol molecules. A preferred compound is a compound of Formula XVIII:

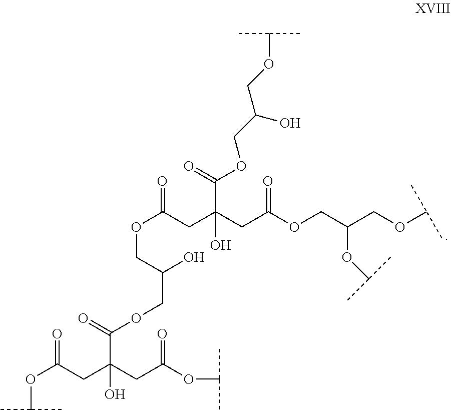

Another aspect of the invention provides methods for treating a condition associated with altered TCA cycle metabolism in a subject comprising providing any of the above described compositions in a therapeutically effective amount to treat the condition associated with altered TCA cycle metabolism in the subject. The condition associated with altered TCA cycle metabolism may be an inherited disorder, a neurodegenerative disorder, a cancer, an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy, mitochondrial myopathy, mitochondrial encephalomyopathy lactic acidosis and stroke-like syndrome (MELAS), myoclonic epilepsy and ragged-red fibers (MERRF), a mitochondrial associated disease, and a disorder related to POLG mutation.

In another aspect, the invention provides compositions comprising a TCA cycle intermediate anhydride or polymer or pharmaceutically acceptable salt or prodrug thereof in a therapeutically effective amount to treat a condition associated with altered TCA cycle metabolism in a subject. The TCA cycle intermediate or pharmaceutically acceptable salt or polymer or prodrug thereof may be selected from the group consisting of citrate, cis-aconitate, D-isocitrate, α-ketoglutarate, succinate, fumarate, malate, oxaloacetate, pyruvate, acetone, acetoacetate, β-hydroxybutyrate, β-ketopentanoate, and β-hydroxypentanoate.

In certain embodiments, the prodrug comprises one or more polyols. In other embodiments, the prodrug comprises one or more amino acids. In certain embodiments, the prodrug comprises one or more polyols and one or more amino acids. In certain embodiments, the composition is a polymer of a TCA cycle intermediate, e.g., one or more repeating units of a TCA cycle intermediate monomer.

In an exemplary embodiment, the TCA cycle intermediate anhydride or polymer or pharmaceutically acceptable salt or prodrug thereof is citric acid anhydride or polymer or a pharmaceutically acceptable salt or prodrug thereof. In certain embodiments, the citric acid anhydride is selected from the group consisting of a symmetrical citric acid anhydride (Formula XIX below), an asymmetrical citric acid anhydride (Formula XX below), an intermolecular citric acid anhydride (Formula XXI below), and a combination thereof.

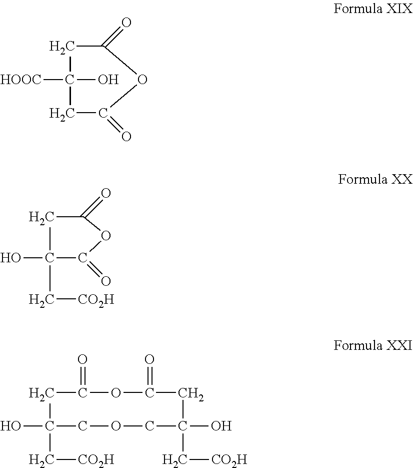

In certain embodiments, the citric acid anhydride is a prodrug of citric acid anhydride. Such exemplary prodrugs may comprise one or more polyols. In other embodiments, the citric acid anhydride prodrug comprises one or more amino acids. In certain embodiments, the citric acid anhydride prodrug comprises one or more polyols and one or more amino acids. In certain embodiments, the composition is a citric acid anhydride polymer, e.g., one or more repeating units of a citric acid anhydride monomer.

In certain embodiments, the composition is formulated for oral or gastric administration. In certain embodiments, the composition is formulated as a single unit dose.

Another aspect of the invention provides methods for treating a condition associated with altered TCA cycle metabolism in a subject comprising providing any of the above described compositions in a therapeutically effective amount to treat the condition associated with altered TCA cycle metabolism in the subject. The condition associated with altered TCA cycle metabolism may be an inherited disorder, a neurodegenerative disorder, a cancer, an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy, mitochondrial myopathy, mitochondrial encephalomyopathy lactic acidosis and stroke-like syndrome (MELAS), myoclonic epilepsy and ragged-red fibers (MERRF), a mitochondrial associated disease, and a disorder related to POLG mutation.

The invention provides pharmaceutical compositions containing one or more of the compounds described above. A pharmaceutical composition containing the compounds may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. 2003/0232877, incorporated by reference herein in their entirety.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water. Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Compositions of the invention may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol, sorbitol, mannitol and polyethylene glycol; esters, such asethyl oleate and ethyllaurate; agar, buffering agents, such as magnesium hydroxide and aluminum hydroxide; alginic acid; pyrogen-free water, isotonic saline; Ringer's solution; ethyl alcohol; pH buffered solutions; polyesters, polycarbonates and/or polyanhydrides; and other non-toxic compatible substances employed in pharmaceutical formulations.

Compounds of the invention may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, β-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

As indicated above, the compounds of the invention allow for delivery of TCA cycle intermediates or prodrugs thereof to tissues that have abnormal TCA cycle metabolism. Thus, the invention also provides methods of treating conditions associated with altered TCA cycle metabolism in a subject by providing compositions of the invention.

The methods include providing a composition of the invention, as described above, to the subject. Providing may include administering the composition to the subject. The composition may be administered by any suitable means, such as orally, intravenously, enterally, parenterally, dermally, buccally, topically (including transdermally), by injection, intravenously, nasally, pulmonarily, and with or on an implantable medical device (e.g., stent or drug-eluting stent or balloon equivalents).

The condition may be any disease or disorder associated with altered TCA cycle metabolism or that can be ameliorated by providing an intermediate of the TCA cycle. For example, the condition may be an inherited disorder, such as 2-oxoglutaric aciduria, fumarase deficiency, or succinyl-CoA synthetase deficiency. The condition may be a neurodegenerative disorder, such as Amyotrophic Lateral Sclerosis. Alzheimer's disease, Parkinson's disease, or Huntington's disease. The condition may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma. The condition may be an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy (e.g., Duchenne's muscular dystrophy and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to a POLG mutation.

### EXAMPLES

### Example 1

The compound of formula (XIII) was made and analyzed. The compound of formula (XIII) was found to have a solubility in water of 1.2 g/mL and to taste slightly sweet but not bitter.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1-95. (canceled)

96. A compound comprising:
one or more TCA cycle intermediates or prodrugs thereof; and
one or more non-naturally-occurring amino acids.

97. The compound of claim 96, wherein the one or more non-naturally-occurring amino acids comprise a non-naturally-occurring derivative of cysteine.

98. The compound of claim 97, wherein the one or more TCA cycle intermediates or prodrugs thereof comprise succinate.

99. The compound of claim 98, wherein the succinate is covalently linked via a terminal carbon of the succinate to the non-naturally-occurring derivative of cysteine.

100. The compound of claim 98, wherein the compound comprises two non-naturally occurring derivatives of cysteine that are the same or different, and wherein the succinate is covalently linked via a first terminal carbon of the succinate to a first of the two non-naturally-occurring derivatives of cysteine and is covalently linked via a second terminal carbon of the succinate to a second of the two non-naturally-occurring derivatives of cysteines

101. The compound of claim 96, wherein the compound further comprises a naturally-occurring amino acid.

102. The compound of claim 101, wherein the naturally-occurring amino acid is serine.

103. The compound of claim 102, wherein the non-naturally-occurring amino acid is a non-naturally-occurring derivative of cysteine.

104. The compound of claim 103, wherein the one or more TCA cycle intermediates or prodrugs thereof comprise succinate.

105. The compound of claim 104, wherein the succinate is covalently linked via a first terminal carbon of the succinate to the serine and is covalently linked via a second terminal carbon of the succinate to the non-naturally-occurring derivative of cysteine.

106. A method of treating a condition associated with altered TCA cycle metabolism in a subject, the method comprising providing to a subject having a condition associated with altered TCA cycle metabolism a compound comprising:
one or more TCA cycle intermediates or prodrugs thereof; and
one or more non-naturally-occurring amino acids.

107. The method of claim 106, wherein the one or more non-naturally-occurring amino acids comprise a non-naturally-occurring derivative of cysteine.

108. The method of claim 107, wherein the one or more TCA cycle intermediates or prodrugs thereof comprise succinate.

109. The method of claim 108, wherein the succinate is covalently linked via a terminal carbon of the succinate to the non-naturally-occurring derivative of cysteine.

110. The method of claim 108, wherein the compound comprises two non-naturally occurring derivatives of cysteine that are the same or different, and wherein the succinate is covalently linked via a first terminal carbon of the succinate to a first of the two non-naturally-occurring derivatives of cysteine and is covalently linked via a second terminal carbon of the succinate to a second of the two non-naturally-occurring derivatives of cysteines

111. The method of claim 106, wherein the compound further comprises a naturally-occurring amino acid.

112. The method of claim 111, wherein the naturally-occurring amino acid is serine.

113. The method of claim 112, wherein the non-naturally-occurring amino acid is a non-naturally-occurring derivative of cysteine.

114. The method of claim 113, wherein the one or more TCA cycle intermediates or prodrugs thereof comprise succinate.

115. The method of claim 114, wherein the succinate is covalently linked via a first terminal carbon of the succinate to the serine and is covalently linked via a second terminal carbon of the succinate to the non-naturally-occurring derivative of cysteine.

116. The method of claim 106, wherein the condition associated with altered TCA cycle metabolism is selected from the group consisting of an inherited disorder, a neurodegenerative disorder, a cancer, an energetic disorder, refractory epilepsy, propionic acidemia (PA), methylmalonic acidemia (MMA), a long chain fatty acid oxidation disorder, succinyl CoA lyase deficiency, pyruvate carboxylase deficiency, mitochondrial respiratory chain deficiency, glutaric acidemia type 1 or type 2 a neurologic disease, disorder or condition, a pain or fatigue disease, muscular dystrophy, mitochondrial myopathy, mitochondrial encephalomyopathy lactic acidosis and stroke-like syndrome (MELAS), myoclonic epilepsy and ragged-red fibers (MERRF), a mitochondrial associated disease, and a disorder related to POLG mutation.
